(19) United States
(12) Patent Application Publication
(10) Pub. No.: US 2010/0152795 A1
(43) Pub. Date: Jun. 17, 2010

(54) IDENTIFICATION OF DYSYNCHRONY USING INTRACARDIAC ELECTROGRAM DATA

(75) Inventor: Stuart O. Schecter, Great Neck, NY (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/332,725

(22) Filed: Dec. 11, 2008

(51) Int. Cl. A61N 1/39; A61N 1/368

(52) U.S. Cl. 607/4; 607/28; 607/32; 607/17

(57) ABSTRACT

Implantable stimulation devices can provide intracardiac electrograms (EGMs) and impedance measurements to detect changes in electrical, mechanical, and electromechanical activation of the heart. Many patients with congestive heart failure have conventional intracardiac devices implanted that are not capable of resynchronization therapy and these patients could benefit from resynchronization, but are not candidates based on current criteria. These patient populations can be identified through analyses of intracardiac electrogram data that is available through implantable stimulation devices comprising at least one lead for providing electrical stimulation to the heart of a patient, at least one sensor that detects electrical signals indicative of the depolarization of the heart of the patient, and a controller that is adapted to be implanted within the patient. The controller receives signals from the at least one sensor and further induces the lead to provide therapeutic electrical stimulation to the heart of the patient. The controller periodically evaluates the signals from the sensor and determines if at least one parameter of the signal is indicative of the patient being potentially subject to heart dysynchrony. The controller, upon determining that the parameter of the signal indicates that the patient is potentially subject to heart dysynchrony, records an indication thereof for subsequent communication to treating medical personnel.

### BACKGROUND OF THE INVENTION

1. Field of the Invention

This invention relates generally to implantable stimulation devices, and more particularly to identifying dysynchrony in patients with implantable stimulation devices.

2. Description of the Related Art

The mechanical events of the heart are preceded and initiated by the electrochemical activity of the heart (i.e., the propagation of the action potential). In a healthy heart, the electrical and mechanical operation of the heart is regulated by electrical signals produced by the heart's sino-atrial (SA) node. Each signal produced by the SA node spreads across the atria, causing the depolarization and contraction of the atria, and arrives at the atrioventicular (A-V) node. The signal is then conducted to the “Bundle of His” during which time it is slowed down to allow for the atrium to pump blood into the ventricles and thereafter to the “Bundle Branches” and the Purkinje muscle fibers of the right and left ventricles. The signals propagated through the Bundle Branches effects depolarization and accompanying contraction of the left ventricle and the right ventricle in close order. Following contraction, the myocardial cells repolarize during a short period of time, returning to their resting state. The right and left atria refill with venous and oxygenated blood, respectively, until the cardiac cycle is again commenced by a signal originating from the SA node. At rest, the normal adult SA node produces a signal approximately 60 to 85 times a minute, causing the heart muscle to contract, and thereby pumping blood to the remainder of the body. The electrical signal passes through the heart chambers as a wave front that can be characterized as a plane advancing from cell to cell through the cardiac muscle that separates cells of different electrical potential as a function of the time that it takes to complete the cardiac cycle.

The above-described cardiac cycle is disrupted in diseased or injured hearts, and the chronic or episodic disrupted electrical activity has long been employed to diagnose the state of the heart. A variety of techniques have been developed for collecting and interpreting data concerning the electrical activity of the heart.

A commonly used technique is the electrocardiograph (ECG) machine that displays one-dimension tracings of electrical signals of the heart as the depolarization wave front advances across the heart chambers in the cardiac cycle. An ECG machine typically measures and displays and/or records the voltages at various skin electrodes placed about the body relative to a designated “ground” electrode. The paired electrodes are referred to as “leads” and the lead signal is displayed or printed as an ECG lead tracing. The term “lead” would appear to indicate a physical wire, but in electrocardiography, “lead” actually means the electrical signal or vector in space between a designated pair of skin electrodes arranged as described below, wherein the vectors traverse the heart disposed between the skin electrodes.

The cardiac cycle as displayed in an ECG lead tracing reflects the electrical wave front as measured across one such ECG lead. The portion of a cardiac cycle representing atrial depolarization is referred to as a “P-wave”. Depolarization of the ventricular muscle fibers is represented by “Q”, “R”, and “S” points of a cardiac cycle. Collectively these “QRS” points are called an “R-wave” or a “QRS complex.” Re-polarization of the depolarized heart cells occurs after the termination of another positive deflection following the QRS complex known as the “T-wave.” The QRS complex is the most studied part of the cardiac cycle and is considered to be important for the prediction of health and survivability of a patient. However, the time relation of the P-wave to the QRS complex and the height and polarity of the T-wave and S-T segment are also indicators of a healthy or diseased heart.

Heart failure affects millions of people. With heart failure, the heart attempts to meet the energy demands of the body and may begin to compensate for lost pumping power. To compensate, the heart muscle can become enlarged and change shape. These changes can result in an uncoordinated (or unsynchronized) and inefficient heartbeat called dysynchrony. With dysynchrony, the chambers of the heart are not effectively synchronized. Dysynchrony can force the heart to work harder which can cause more heart failure symptoms.

Heart failure can cause electrical abnormalities in the conduction of the electrical signals that stimulate the heart to contract and pump blood. Scarred heart tissue resulting from heart failure, for example, can cause a disruption in the conduction patterns of the heart's electrical signals. In a normal heart, the electrical signals travel to the heart muscle and cause the heart to contract synchronously and in a symmetric fashion in relation to the opening and closing of the heart valves. Delays in the conduction or an alternate conduction path of the electrical signals due to scar tissue can result in dysynchrony.

Heart failure can also cause mechanical abnormalities in the ability of the heart to contract. Scarred heart tissue resulting from heart failure may not contract at the same rate or as much as normal heart tissue. This also can be a cause of dysynchrony.

Whereas it was once thought that the prolongation of electrical signals, such as the duration of the QRS segment as demonstrated by a surface electrocardiogram was a specific indication of dysynchrony, more recent data supports that this is not necessarily accurate. Recent publications have confirmed a lack of specificity and/or sensitivity of using the QRS width in determining dysynchrony. For example, patients having infranodal conduction abnormalities, such as syncope or intermittent heart block, have narrow QRS patterns and dysynchrony. These patients would not be considered as having dysynchrony based on the current guideline.

People with heart failure and dysynchrony can benefit from cardiac resynchronization therapy (CRT). Cardiac Resynchronizing Therapy (CRT) refers to pacing techniques to ater the degree of electromechanical asynchrony in patients with conduction disorders. Pacing to coordinate the contraction of the ventricles or atrials can use current implantable stimulation devices by incorporating pacing.

Many patients with heart failure are already implanted with implantable stimulation devices, such as such as automatic implantable cardiac defibrillators (ICDs), stand-alone biventricular pacemakers, tachycardia pacemakers, bradycardia pacemakers, or the like, to stimulate the heart. Implantable stimulation devices comprise leads to stimulate the heart and sensors to record the electrical activity of the heart. Current implantable stimulation devices also allow physicians to assess intracardiac electrograms (EGMs) and impedance measurements recorded by the sensors and detect changes in electrical, mechanical, and electromechanical activation of the heart not apparent on surface ECGs. However, many patients with congestive heart failure having conventional intracardiac devices implanted can benefit from resynchronization, but are not candidates based on current criteria.

### SUMMARY OF THE INVENTION

Implantable stimulation devices can provide intracardiac electrograms (EGMs) and impedance measurements to detect changes in electrical, mechanical, and electromechanical activation of the heart. A patient, where the patient is a mammalian body and preferably a human body, can be identified as having dysynchrony through analysis of this data. The implantable stimulation device can record an indication of possible dysynchrony for subsequent communication to treating medical personnel.

In an embodiment, an implantable cardiac stimulation device comprises at least one lead for providing electrical stimulation to the heart of a patient, at least one sensor that detects electrical signals indicative of the depolarization of the heart of the patient, and a controller that is adapted to be implanted within the patient. The controller receives signals from the at least one sensor and further induces the lead to provide therapeutic electrical stimulation to the heart of the patient. The controller periodically evaluates the signals from the sensor and determines if at least one parameter of the signal is indicative of the patient being potentially subject to heart dysynchrony. The controller, upon determining that the parameter of the signal indicates that the patient is potentially subject to heart dysynchrony, records an indication thereof for subsequent communication to treating medical personnel.

In another embodiment, an implantable cardiac stimulation device comprises a means for providing electrical stimulation to the heart of a patient, a means for detecting electrical signals indicative of the depolarization of the heart of the patient, and a means for processing that is adapted to be implanted within the patient. The means for processing receives signals from the means for detecting and further induces the means for providing to provide therapeutic electrical stimulation to the heart of the patient. The means for processing periodically evaluates the signals from the means for detecting and determines if at least one parameter of the signal is indicative of the patient being potentially subject to heart dysynchrony. The means for processing, upon determining that the parameter of the signal indicates that the patient is potentially subject to heart dysynchrony, records an indication thereof for subsequent communication to treating medical personnel.

In yet another embodiment, a method for using an implantable cardiac stimulation device comprises providing electrical stimulation to the heart of a patient, detecting electrical signals indicative of the depolarization of the heart of the patient, receiving the electrical signals with a controller adapted to be implanted within the patient, and providing therapeutic electrical stimulation to the heart of the patient based at least in part on the received electrical signals. The method further comprises periodically evaluating the signals with the controller, and determining with the controller whether at least one parameter of the signal is indicative of the patient being potentially subject to heart dysynchrony. Upon determining that the parameter of the signal indicates that the patient is potentially subject to heart dysynchrony, the method further comprises recording with the controller an indication thereof for subsequent communication to treating medical personnel.

For purposes of summarizing the invention, certain aspects, advantages, and novel features of the invention have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

Further features and advantages of the present invention may be more readily understood by reference to the following description taken in conjunction with the accompanying drawings, in which:

FIG. 1 is a simplified diagram illustrating an implantable stimulation device in electrical communication with at least three leads implanted into a patient's heart for delivering multi-chamber stimulation and shock therapy, according to an embodiment of the invention;

FIG. 2 is a functional block diagram of a multi-chamber implantable stimulation device illustrating the basic elements of a stimulation device, which can provide cardioversion, defibrillation and pacing stimulation in four chambers of the heart, according to an embodiment of the invention;

FIG. 3 is a functional block diagram of an external device, according to an embodiment of the device.

FIG. 4 is a flow chart describing an overview of the operation for identifying dysynchrony in patients, according to an embodiment of the invention;

FIG. 5 is a schematic illustration of an exemplary implantable stimulation device and lead system for deriving a plurality of EGM vector signals.

FIG. 6 is illustrates exemplary data used to derive dysynchrony indices.

FIG. 7 illustrates time dependent curves of impedance at two locations in the patient's heart and the patient's intracardiac electrogram, according to an embodiment of the invention.

FIG. 8 is a schematic illustration of a dysynchrony calculator, according to an embodiment of the invention.

FIG. 9 is a functional block diagram of a system for determining dysynchrony, according to an embodiment of the invention.

FIG. 10 is an exemplary intracardiac electrogram of a first patient;

FIG. 11 is an exemplary intracardiac electrogram of a second patient.

### DETAILED DESCRIPTION OF THE PREFERRED EMBODIMENT

The following description is of the best mode presently contemplated for practicing the invention. This description is not to be taken in a limiting sense but is made merely for the purpose of describing the general principles of the invention. The scope of the invention should be ascertained with reference to the issued claims. In the description of the invention that follows, like numerals or reference designators will be used to refer to like parts or elements throughout.

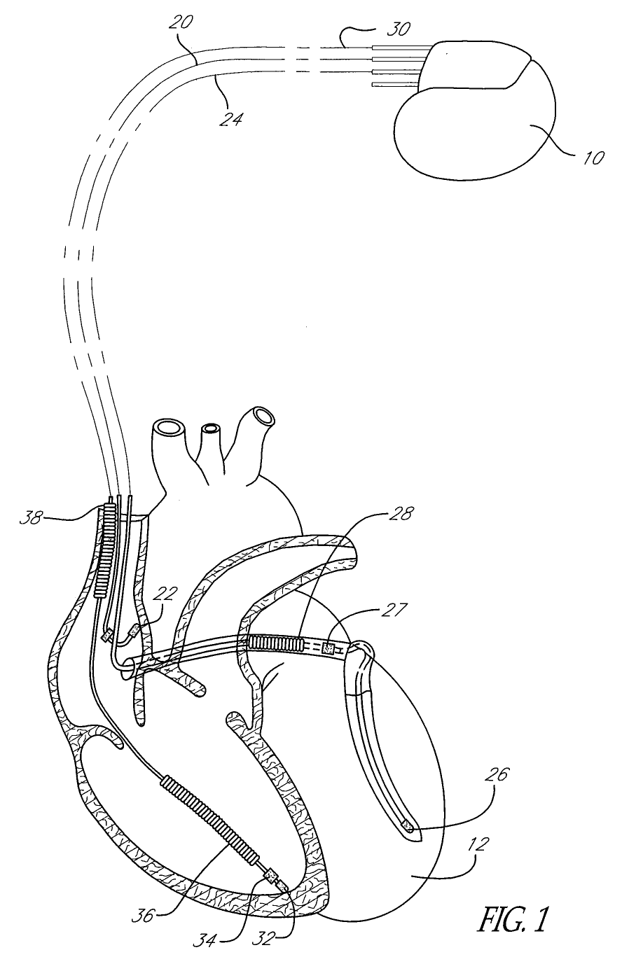

As shown in FIG. 1, there is a stimulation device 10 in electrical communication with a patient's heart 12 by way of three leads, 20, 24 and 30, suitable for delivering multi-chamber stimulation and shock therapy. To sense atrial cardiac signals and to provide right atrial chamber stimulation therapy, the stimulation device 10 is coupled to an implantable right atrial lead 20 having at least an atrial tip electrode 22, which typically is implanted in the patient's right atrial appendage.

To sense left atrial and ventricular cardiac signals and to provide left chamber pacing therapy, the stimulation device 10 is coupled to a “coronary sinus” lead 24 designed for placement in the “coronary sinus region” via the coronary sinus os for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, an exemplary coronary sinus lead 24 is designed to receive atrial and ventricular cardiac signals and to deliver left ventricular pacing therapy using at least a left ventricular tip electrode 26, left atrial pacing therapy using at least a left atrial ring electrode 27, and shocking therapy using at least a left atrial coil electrode 28. For a complete description of a coronary sinus lead, see U.S. patent application Ser. No. 09/196,898, “A Self-Anchoring Coronary Sinus Lead” (Pianca et al.), and U.S. Pat. No. 5,466,254, “Coronary Sinus Lead with Atrial Sensing Capability” (Helland), which patents are hereby incorporated herein by reference.

The stimulation device 10 is also shown in electrical communication with the patient's heart 12 by way of an implantable right ventricular lead 30 having, in this embodiment, a right ventricular tip electrode 32, a right ventricular ring electrode 34, a right ventricular (RV) coil electrode 36, and an SVC coil electrode 38. Typically, the right ventricular lead 30 is transvenously inserted into the heart 12 so as to place the right ventricular tip electrode 32 in the right ventricular apex so that the RV coil electrode 36 will be positioned in the right ventricle and the SVC coil electrode 38 will be positioned in the superior vena cava. Accordingly, the right ventricular lead 30 is capable of receiving cardiac signals, and delivering stimulation in the form of pacing and shock therapy to the right ventricle.

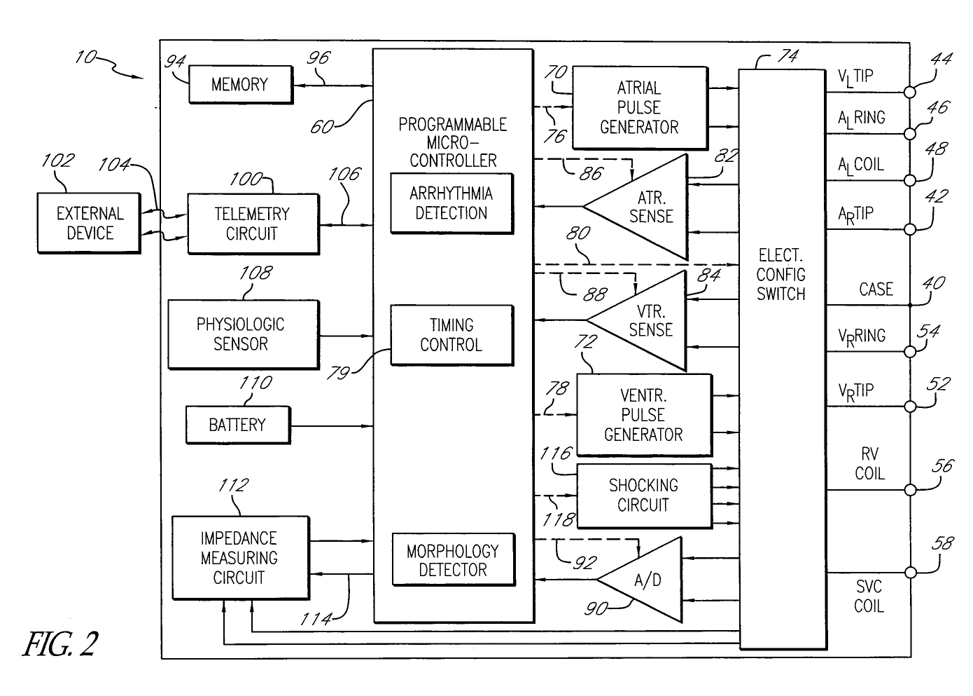

As illustrated in FIG. 2, a simplified block diagram is shown of the multi-chamber implantable stimulation device 10, which is capable of treating both fast and slow arrhythmias with stimulation therapy, such as cardioversion, defibrillation, and pacing stimulation. While a particular multi-chamber device is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation.

The housing 40 for the stimulation device 10, shown schematically in FIG. 2, is often referred to as the “can”, “case”, or “case electrode” and can be programmably selected to act as the return electrode for all “unipolar” modes. The housing 40 can further be used as a return electrode alone or in combination with one or more of the coil electrodes, 28, 36 and 38, for shocking purposes. The housing 40 further comprises a connector (not shown) having a plurality of terminals, 42, 44, 46, 48, 52, 54, 56, and 58 (shown schematically and, for convenience, the names of the electrodes to which they are connected are shown next to the terminals). As such, to achieve right atrial sensing and pacing, the connector comprises at least a right atrial tip terminal (AR TIP) 42 adapted for connection to the atrial tip electrode 22.

To achieve left chamber sensing, pacing and shocking, the connector comprises at least a left ventricular tip terminal (VL TIP) 44, a left atrial ring terminal (AL RING) 46, and a left atrial shocking terminal (AL COIL) 48, which are adapted for connection to the left ventricular tip electrode 26, the left atrial ring electrode 27, and the left atrial coil electrode 28, respectively.

To support right chamber sensing, pacing and shocking, the connector further comprises a right ventricular tip terminal (VR TIP) 52, a right ventricular ring terminal (VR RING) 54, a right ventricular shocking terminal (RV COIL) 56, and an SVC shocking terminal (SVC COIL) 58, which are adapted for connection to the right ventricular tip electrode 32, right ventricular ring electrode 34, the RV coil electrode 36, and the SVC coil electrode 38, respectively.

At the core of the stimulation device 10 is a programmable microcontroller 60, which controls the various modes of stimulation therapy. As is well known in the art, the microcontroller 60 typically comprises a microprocessor, or equivalent control circuitry, designed specifically for controlling the delivery of stimulation therapy and can further include RAM or ROM memory, logic and timing circuitry, state machine circuitry, and I/O circuitry. Typically, the microcontroller 60 comprises the ability to process or monitor input signals (data) as controlled by a program code stored in a designated block of memory. The details of the design and operation of the microcontroller 60 are not critical to the present invention. Rather, any suitable microcontroller 60 can be used that carries out the functions described herein. The use of microprocessor-based control circuits for performing timing and data analysis functions are well known in the art.

As shown in FIG. 2, an atrial pulse generator 70 and a ventricular pulse generator 72 generate pacing stimulation pulses for delivery by the right atrial lead 20, the right ventricular lead 30, and/or the coronary sinus lead 24 via an electrode configuration switch 74. It is understood that in order to provide stimulation therapy in each of the four chambers of the heart, the atrial and ventricular pulse generators, 70 and 72, can include dedicated, independent pulse generators, multiplexed pulse generators, or shared pulse generators. The pulse generators, 70 and 72, are controlled by the microcontroller 60 via appropriate control signals, 76 and 78, respectively, to trigger or inhibit the stimulation pulses.

The microcontroller 60 further comprises timing control circuitry 79 which is used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, PVARP intervals, noise detection windows, evoked response windows, alert intervals, marker channel timing, etc., which is well known in the art.

The switch 74 comprises a plurality of switches for connecting the desired electrodes to the appropriate I/O circuits, thereby providing electrode programmability. Accordingly, the switch 74, in response to a control signal 80 from the microcontroller 60, determines the polarity of the stimulation pulses (e.g., unipolar, bipolar, combipolar, etc.) by selectively closing the appropriate combination of switches (not shown) as is known in the art.

Atrial sensing circuits 82 and ventricular sensing circuits 84 can also be selectively coupled to the right atrial lead 20, coronary sinus lead 24, and the right ventricular lead 30, through the switch 74 for detecting the presence of cardiac activity in each of the four chambers of the heart. Accordingly, the atrial (ATR. SENSE) and ventricular (VTR. SENSE) sensing circuits, 82 and 84, can include dedicated sense amplifiers, multiplexed amplifiers, or shared amplifiers. The switch 74 determines the “sensing polarity” of the cardiac signal by selectively closing the appropriate switches, as is also known in the art. In this way, the clinician can program the sensing polarity independent of the stimulation polarity.

Each sensing circuit, 82 and 84, preferably employs one or more low power, precision amplifiers with programmable gain and/or automatic gain control, bandpass filtering, and a threshold detection circuit, as known in the art, to selectively sense the cardiac signal of interest. The automatic gain control enables the device 10 to deal effectively with the difficult problem of sensing the low amplitude signal characteristics of atrial or ventricular fibrillation. The outputs of the atrial and ventricular sensing circuits, 82 and 84, are connected to the microcontroller 60 which, in turn, are able to trigger or inhibit the atrial and ventricular pulse generators, 70 and 72, respectively, in a demand fashion in response to the absence or presence of cardiac activity in the appropriate chambers of the heart. The sensing circuits, 82 and 84, in turn, receive control signals over signal lines, 86 and 88, from the microcontroller 60 for purposes of controlling the gain, threshold, polarization charge removal circuitry (not shown), and the timing of any blocking circuitry (not shown) coupled to the inputs of the sensing circuits, 82 and 86, as is known in the art.

For arrhythmia detection, the device 10 utilizes the atrial and ventricular sensing circuits, 82 and 84, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation which are sometimes referred to as “F-waves” or “Fib-waves”) are then classified by the microcontroller 60 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”).

Cardiac signals are also applied to the inputs of an analog-to-digital (A/D) data acquisition system 90. The data acquisition system 90 is configured to acquire intracardiac electrogram (EGM) signals, convert the raw analog data into a digital signal, and store the digital signals for later processing and/or telemetric transmission to an external device 102. The data acquisition system 90 is coupled to the right atrial lead 20, the coronary sinus lead 24, and the right ventricular lead 30 through the switch 74 to sample cardiac signals across any pair of desired electrodes.

Advantageously, the data acquisition system 90 can be coupled to the microcontroller, or other detection circuitry, for detecting an evoked response from the heart 12 in response to an applied stimulus, thereby aiding in the detection of “capture”. Capture occurs when an electrical stimulus applied to the heart is of sufficient energy to depolarize the cardiac tissue, thereby causing the heart muscle to contract. The microcontroller 60 detects a depolarization signal during a window following a stimulation pulse, the presence of which indicates that capture has occurred. The microcontroller 60 enables capture detection by triggering the ventricular pulse generator 72 to generate a stimulation pulse, starting a capture detection window using the timing control circuitry 79 within the microcontroller 60, and enabling the data acquisition system 90 via control signal 92 to sample the cardiac signal that falls in the capture detection window and, based on the amplitude, determines if capture has occurred.

Capture detection can occur on a beat-by-beat basis or on a sampled basis. Preferably, a capture threshold search is performed once a day during at least the acute phase (e.g., the first 30 days) and less frequently thereafter. A capture threshold search would begin at a desired starting point (either a high energy level or the level at which capture is currently occurring) and decrease the energy level until capture is lost. The value at which capture is lost is known as the capture threshold. Thereafter, a safety margin is added to the capture threshold.

The microcontroller 60 is further coupled to a memory 94 by a suitable data/address bus 96, wherein the programmable operating parameters used by the microcontroller 60 are stored and modified, as required, in order to customize the operation of the stimulation device 10 to suit the needs of a particular patient. Such operating parameters define, for example, pacing pulse amplitude, pulse duration, electrode polarity, rate, sensitivity, automatic features, arrhythmia detection criteria, and the amplitude, waveshape, and vector of each shocking pulse to be delivered to the patient's heart 12 within each respective tier of therapy. An embodiment of the invention senses and stores a relatively large amount of data (e.g., from the data acquisition system 90), which data can then be used for subsequent analysis to guide the programming of the device 10.

Advantageously, the operating parameters of the implantable device 10 can be non-invasively programmed into the memory 94 through a telemetry circuit 100 in telemetric communication with the external device 102, such as a programmer, transtelephonic transceiver, or a diagnostic system analyzer. The telemetry circuit 100 is activated by the microcontroller by a control signal 106. The telemetry circuit 100 advantageously allows intracardiac electrograms and status information relating to the operation of the device 10 (as contained in the microcontroller 60 or memory 94) to be sent to the external device 102 through an established communication link 104.

In the preferred embodiment, the stimulation device 10 further comprises a physiologic sensor 108, commonly referred to as a “rate-responsive” sensor because it is typically used to adjust pacing stimulation rate according to the exercise state of the patient. However, the physiological sensor 108 can further be used to detect changes in cardiac output, changes in the physiological condition of the heart, or diurnal changes in activity (e.g., detecting sleep and wake states). Accordingly, the microcontroller 60 responds by adjusting the various pacing parameters (such as rate, AV Delay, V-V Delay, etc.) at which the atrial and ventricular pulse generators, 70 and 72, generate stimulation pulses. While shown as being included within the stimulation device 10, it is to be understood that the physiologic sensor 108 can also be external to the stimulation device 10, yet still be implanted within or carried by the patient. A common type of rate responsive sensor is an activity sensor, such as an accelerometer or a piezoelectric crystal, which is mounted within the housing 40 of the stimulation device 10. Other types of physiologic sensors are also known, for example, sensors, which sense the oxygen content of blood, respiration rate and/or minute ventilation, pH of blood, ventricular gradient, etc. However, any sensor can be used which is capable of sensing a physiological parameter which corresponds to the exercise state of the patient. The type of sensor used is not critical and is shown only for completeness.

The stimulation device additionally comprises a battery 110, which provides operating power to the circuits shown in FIG. 2. For the stimulation device 10, which employs shocking therapy, the battery 110 is capable of operating at low current drains for long periods of time, and then be capable of providing high-current pulses (for capacitor charging) when the patient requires a shock pulse. The battery 110 also has a predictable discharge characteristic so that elective replacement time can be detected. Accordingly, the device 10 preferably employs lithium/silver vanadium oxide batteries.

The stimulation device 10 further comprises a magnet detection circuitry (not shown), coupled to the microcontroller 60. It is the purpose of the magnet detection circuitry to detect when a magnet is placed over the stimulation device 10, which magnet can be used by a clinician to perform various test functions of the stimulation device 10 and/or to signal the microcontroller 60 that the external programmer 102 is in place to receive or transmit data to the microcontroller 60 through the telemetry circuits 100.

As further shown in FIG. 2, the device 10 is shown as having an impedance measuring circuit 112, which is enabled by the microcontroller 60 via a control signal 114. The known uses for an impedance measuring circuit 112 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 112 is advantageously coupled to the switch 74 so that any desired electrode can be used.

The programmable microcontroller can further comprise a dysynchrony calculator 810 configured to analyze the EGM data, the impedance data, and the pacing frequency, alone or in combination, to determine whether an indication of dysynchrony exists.

In the case where the stimulation device 10 is intended to operate as an implantable cardioverter/defibrillator (ICD) device, it detects the occurrence of an arrhythmia, and automatically apply an appropriate electrical shock therapy to the heart aimed at terminating the detected arrhythmia. To this end, the microcontroller 60 further controls a shocking circuit 116 by way of a control signal 118. The shocking circuit 116 generates shocking pulses of low (up to 0.5 Joules), moderate (0.5-10 Joules), or high energy (11 to 40 Joules), as controlled by the microcontroller 60. Such shocking pulses are applied to the patient's heart 12 through at least two shocking electrodes, and as shown in this embodiment, selected from the left atrial coil electrode 28, the RV coil electrode 36, and/or the SVC coil electrode 38. As noted above, the housing 40 can act as an active electrode in combination with the RV electrode 36, or as part of a split electrical vector using the SVC coil electrode 38 or the left atrial coil electrode 28 (i.e., using the RV electrode as a common electrode).

Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 5-40 Joules), delivered asynchronously (since R-waves may be too disorganized), and pertaining exclusively to the treatment of fibrillation. Accordingly, the microcontroller 60 is capable of controlling the synchronous or asynchronous delivery of the shocking pulses.

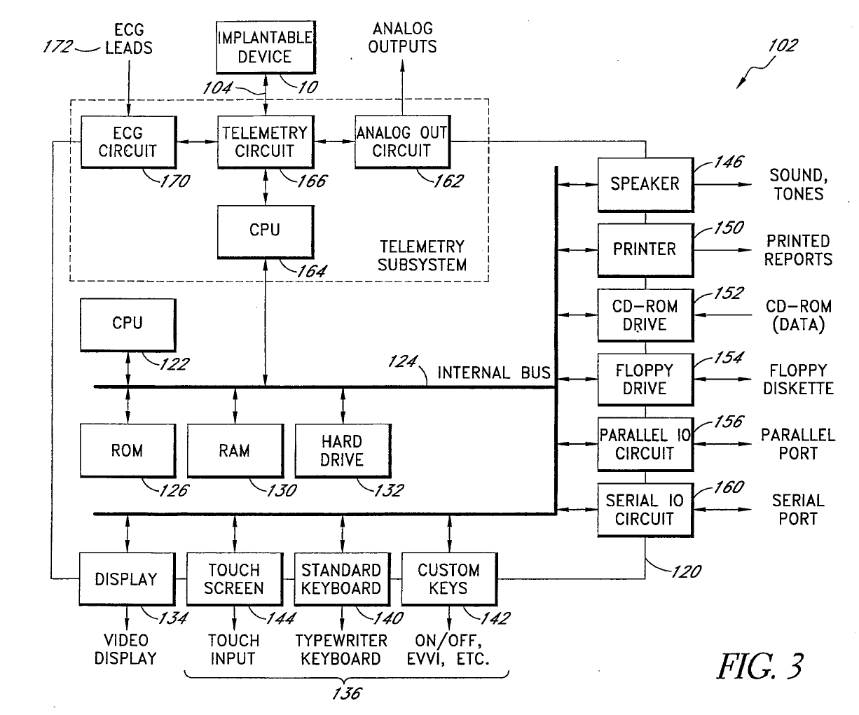

FIG. 3 is a functional block diagram of one embodiment of the external device 102, such as a physician's programmer. The external device 102 comprises a CPU 122 in communication with an external bus 124. The internal bus 124 provides a common communication link and power supply between various electrical components of the external device 102, such as the CPU 122. The external device 102 also comprises memory and data storage such as ROM 126, RAM 130, and a hard drive 132 commonly in communication with the internal bus 124. The ROM 126, RAM 130, and hard drive 132 provide temporary memory and non-volatile storage of data in a well known manner. In one embodiment, the ROM 126, RAM 130, and hard drive 132 can store control programs and commands for upload to the implantable device 10 as well as operating software for display of data received from the implantable device 10. It will be appreciated that in certain embodiments alternative data storage/memory devices, such as flash memory, can be included or replaced one or more of the ROM 126, RAM 130, and hard drive 132 without detracting from the spirit of the invention.

The external device 102 also comprises a display 134. The display 134 is adapted to visually present graphical and alphanumeric data in a manner well understood in the art. The external device 102 also comprises input devices 136 to enable a user to provide commands and input data to the external device 102. In one embodiment, the input devices 136 include a keyboard 140, a plurality of custom keys 142, and a touch screen 144 aspect of the display 134. The keyboard 140 facilitates entry of alphanumeric data into the external device 102. The custom keys 142 are programmable to provide one touch functionality of predefined functions and/or operations. The custom keys 142 can be embodied as dedicated touch keys, such as associated with the keyboard 140 and/or predefined areas of the touch screen 144. In this embodiment, the external device 102 also comprises a speaker 146 and a printer 150 in communication with the internal bus 124. The speaker 146 is adapted to provide audible alert send signals to a user. The printer 150 is adapted to provide a printed readout of information from the external device 102.

In this embodiment, the external device also comprises a CD drive 152 and a floppy drive 154 which together provide removable data storage. In this embodiment, the external device also comprises a parallel input-output (IO) circuit 156, a serial 10 circuit 160, and an analog output circuit 162. These circuits 156, 160, 162 provide a variety of communication capabilities between the external device 102 and other devices in a manner well understood in the art.

The external device 102 also comprises an electrocardiogram (ECG) circuit 170 in communication with a plurality of ECG leads 172. The ECG circuit 170 and the ECG leads 172 obtain electrical signals from the surface of a patient's body and configure the signals for display as an ECG waveform on the display 134 of the external device 102.

The external device 102 also comprises a telemetry CPU 164 and a telemetry circuit 166 which establish the telemetric link 104 in cooperation with the implantable device 10. The telemetric link 104 comprises a bidirectional link to enable the external device 102 and the implantable device 10 to exchange data and/or commands. As previously noted, the establishment of the telemetric link 104 is in certain embodiments facilitated by a wand or programmer head, which is placed in proximity to the implantable device 10. The wand or programmer head facilitates establishment of the telemetric link 104 by placing an antenna structure in a closer proximity to the implantable device 10 to facilitate conduction of transmitted signals to the external device 102.

The telemetric link 104 can comprise a variety of communication protocols appropriate to the needs and limitations of a given application. In certain embodiments, the telemetric link 104 comprises radio frequency (RF) telemetry. In one particular embodiment, the telemetric link 104 comprises a frequency modulated digital communication scheme wherein logic ones are transmitted at a first frequency A and logic zeros are transmitted second frequency B. As the implantable device 10 is powered by a battery having limited capacity and in certain embodiments the external device 102 is powered by line voltage, e.g., not subject to the stringent power limitations of the implantable device 10, the bidirectional telemetric link 104 can proceed in an asymmetric manner. For example, in one embodiment, a transmission power and data rate from the external device 102 to the implantable device 10 via the telemetric link 104 can proceed at higher power levels and/or higher data transmission rates than the reciprocal data rates and transmission power from the implantable device 10 to the external device 102. The telemetry circuit 100 of the implantable device 10 as well as the telemetry circuit 166 and CPU 164 of the external device 102 can select or be adjusted to provide a desired communication protocol and transmission power

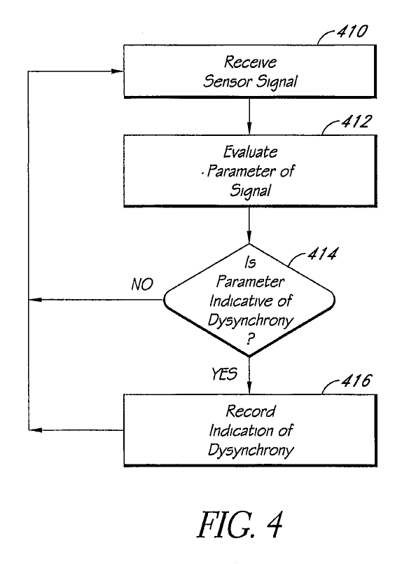

In FIG. 4, a flow chart 400 is shown describing an overview of the operation and novel features implemented in one embodiment of the invention. In the flow chart 400, the various algorithmic steps are summarized in individual “blocks”. Such blocks describe specific actions or decisions that are made or carried out as the algorithm proceeds.

In an embodiment where a microcontroller 60 (or equivalent) is employed, the flow chart presented herein provide the basis for a “control program” that can be used by such a microcontroller 60 (or equivalent) to effectuate the desired control of the stimulation device 10. Those skilled in the art may readily write such a control program based on the flow chart 400 and other descriptions presented herein.

FIG. 4 describes an overview of the operation for identifying dysynchrony in patients, according to an embodiment of the invention. In block 410, the microcontroller 60 receives at least one signal indicative of intracardiac electrogram data and measurements of cardiac impedance as a function of time from the implanted sensors 22, 26, 27, 28, 32, 34, 36, 38 and the impedance measurement circuit 112. The EGM and impedance data is collected at periodic intervals, random intervals, during office visits, or the like.

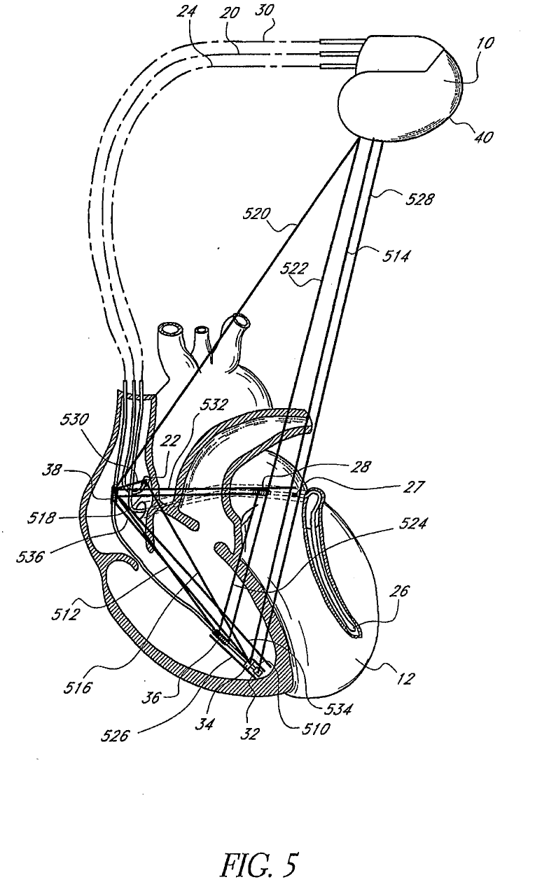

FIG. 5 illustrates examples of some of the possible vectors that can be measured by the device 10 to acquire signals indicative of intra electrocardiogram data and impedance measurements. The vectors represent the depolarization of the heart muscle during a cardiac cycle with respect to time. The vectors are measured between two electrode/sensors 22, 26, 27, 28, 32, 34, 36, 38 or between an electrode/sensor 22, 26, 27, 28, 32, 34, 36, 38 and the case or can 40. The possible vectors include, but are not limited to, a vector between the RV tip sensor 32 and the RV ring sensor 34, referred to as an RV bipolar vector 510; and a vector between the RV coil sensor 36 and the SVC coil 38, referred to as a shock vector 512. Other possible vectors include, but are not limited to a vector 522 between the RV coil sensor 36 and the can 40; a vector 528 between the RV tip sensor 32 and the can 40; and a vector 514 between the RV ring sensor 34 and the case 40. Vectors 522, 528, 514 are examples of surrogate LV vectors that reflect activation or depolarization of left ventricular tissue in the implantable cardiac stimulation device 10 without a left ventricular lead.

In addition, other EGM and impedance measurements are possible from additional vectors, such as, but not limited to a vector 516 between the RV ring sensor 34 and the RA tip sensor 22; a vector 518 between the SVC coil sensor 38 and the LA coil sensor 28; a vector 520 between the SVC coil sensor 38 and the can 40; a vector 524 between the RV ring sensor 36 and the LA coil sensor 28; a vector 526 between the RV ring sensor 34 and the RV coil sensor 36; a vector 530 between the SVC coil sensor 38 and the RA tip sensor 22; a vector 532 between the SVC coil sensor 38 and the LA ring sensor 27; a vector 534, between the SVC coil sensor 38 and the RV tip sensor 32; a vector 536 between the SVC coil sensor 38 and the RV ring sensor 34; and the like. Vectors 512, 518, 520, 530, 532, 534, 536 are examples of surrogate right atrial vectors that reflect the onset of atrial systole in a single chamber implantable cardiac device 10 without a right atrial lead.

Referring to FIG. 4, in block 412, the microcontroller 60 evaluates parameters of the signals. The EGM parameters can comprise intracardiac EGM width in each vector, relative disparity in EGM width between each vector, and comparison of each intracardiac EGM to a normal template. In an embodiment, data for the normal template can be obtained from patients without structural heart disease and ICD implants. The EGM data parameters can further comprise changes in EGM width between periods of selected monitoring; a duration of EGM time above and below baseline; a delay time from intracardiac “P” wave, measured using, for example, the atrial tip electrode 22 or SVC coil electrode 38 to EGM onset in each vector; a delay between EGM onset between the vectors; a delay between intracardiac “P” wave or EGM onset in multiple vectors and timing of EGM termination in multiple vectors; time series analysis of EGM signals; integral data of EGM signals above and below baseline and/or above baseline where negatively deflected EGM data is inverted in a positive direction; data related to time to and time between EGM peak(s) in each vector, and the like.

In addition, the EGM data parameters can comprise a total EGM width (TEW), as measured between the onset of the “P” wave and a terminal end of a left ventricular (LV) vector. In an embodiment, the terminal end of the LV vector can be the time that the amplitude of the EGM signal, post-depolarization, becomes or approaches being isoelectric. In an embodiment, the time that the amplitude of the EGM signal becomes isoelectric can be the time when the change in voltage as a function of time (dV/dt) is at a minima, or when dV′/dt approaches or equals zero. In another embodiment, the time that the amplitude of the EGM signal approaches being isoelectric can be the time when the change in voltage as a function of time (dV/dt) is below a threshold.

Thus, the dysynchrony calculator 810 can measure the total EGM width using an RA bipolar lead or surrogate RV vector 512, 518, 520, 530, 532, 534, 536 and any LV vector in a cardiac resynchronization therapy (CRT) device 10 to alert the attending physician that changes in the timing interval, such as AV/PV delay, RV-LV offset, or the like, can provide improvement to the patient. Similarly, the dysynchrony calculator 810 can measure the total EGM width using the RA bipolar lead or surrogate RV vector 512, 518, 520, 530, 532, 534, 536 and the LV surrogate vector 522, 428, 514 in a non-CRT device 10 to alert the attending physician to consider upgrading the device 10 on one capable of CRT.

In another embodiment, statistical analyses incorporating pattern recognition of acquired EGM templates from exemplary patient groups can be used to identify which patients have dysynchronous activation patterns. For example, tissue synchronization imaging or other acquisition techniques are used to identify dysynchronous patterns in the exemplary patient groups. In addition, surface ECGs and EGM data are acquired from the exemplary patient groups. In an embodiment, the exemplary patient groups are as follows:
Patient Group 1: surface ECG QRS <120 msec, Class 3 or 4 CHF
Patient Group 2: surface ECG QRS ≧120, <150 msec, Class 3 or 4 CHF
Patient Group 3: surface ECG QRS ≧150 msec, Class 3 or 4 CHF
Patient Group 4: surface ECG QRS ≧150 msec, Class 1 or 2 CHF
Patient Group 5: surface ECG any morphology, ejection fraction ≧50%, CHF symptoms, negative ischemia work-up, normal pulmonary function studies, implanted device
Patient Group 6: surface EKG QRS <120 msec, normal EKG, no structural heart disease, no CHF symptoms

In an embodiment, Patient Groups 1-4 have cardiomyopathy with any measured ejection fraction ≦40%. Patient Group 5 is used to assess diastolic function and rule out dysynchrony. Patient Group 6 provides a basis for creating a normal EGM template.

The EGM data associated with the patient groups exhibiting dysynchrony, as determined from the tissue synchronization imaging or other acquisition techniques, are statistically compared by the patient's implanted cardiac device 10 with the patient's EGM data. In an embodiment, the device 10 compares the pattern of the patient's EGM data with the pattern of the Patient Groups' EGM data using a pattern recognition process. In an embodiment, the device 10 uses statistical analysis, such as ANalysis Of VArience (ANOVA) between groups or the like, to determine the statistical significance of the pattern recognition between the patient any of Patient Groups 1-6. Statistical significance between the patient's EGM data and any of the Patient Groups exhibiting dysynchrony indicates the patient also has dysynchrony.

In an embodiment, the EGM signals can comprise repolarization signals. In another embodiment, the duration of the EGM signals can be normalized to the surface ECG QRS complex duration and compared to a threshold value.

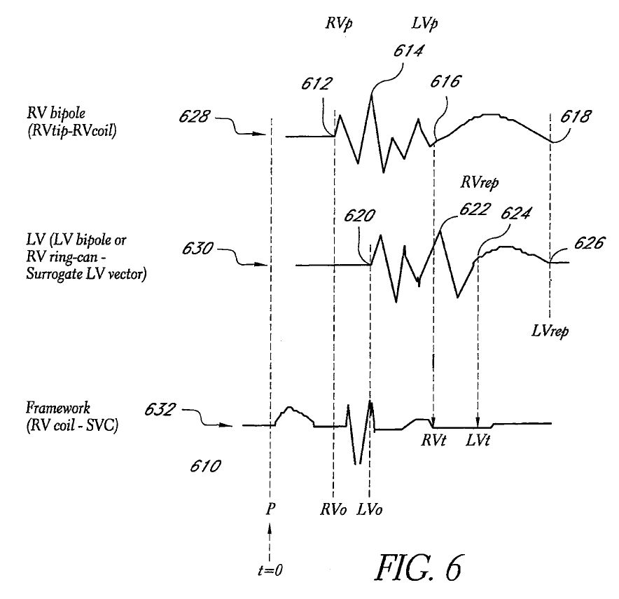

FIG. 6 illustrates examples of EGM waveforms 628, 630, 632 and parameters 612-626 that can be evaluated to determine whether the patient has the possibility of dysynchrony. The device 10 measures a RV bipolar EGM waveform 628, corresponding to the vector 526, between the RV tip sensor 32 and the RV coil sensor 36. The RV bipolar waveform 628 starts a time RV0 612 and indicates the peak of RV depolarization 614, the approximate end of the RV EGM 616, and the approximate end of RV repolarization 618. The device 10 measures a LV EGM waveform 630, corresponding to the vector 514, between the RV ring sensor 34 and the can 40. The LV EGM waveform 630 starts at a time LV0 620 and indicates the peak of LV depolarization 622, the approximate end of the LV EGM 624, and the approximate end of LV repolarization 626. The device 10 measures a shock EGM waveform 632, corresponding to the vector 12, between the RV coil sensor 36 and the SVC coil sensor 38. The shock EGM waveform starts at time 610 and indicates the approximate beginning of the patient's P wave 610.

As shown in FIG. 5, the vectors 512-526 can also be used to acquire impedance measurements using at least one electrode 22, 26, 27, 28, 32, 34, 36, 38 and the impedance measurement circuit 112 in the device 10. In an embodiment, a current is applied between one electrode and a reference electrode and the corresponding induced voltage is measured at a second set of electrodes. For example, data acquisition can be accomplished by delivering pulses of 200 μA having a 30 μsec pulse width at a frequency of 128 Hz to two electrodes 32, 34, positioned along one vector 510/512 and measuring the resulting voltage at electrodes 36 38 located along the same vector 510/512. The resultant time dependent impedance signal, Z(t) peaks when there is maximal systolic ventricular wall thickness and minimal intracardiac blood volume.

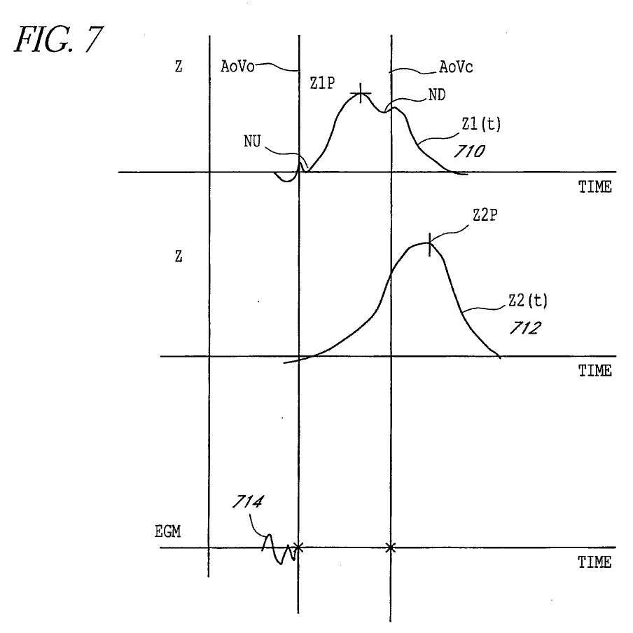

Similar impedance curves can be generated between different electrodes attached to various myocardial segments. FIG. 7 illustrates time dependent curves of impedance Z1(t) 710, Z2(t) 712 at two locations in the patient's heart. The patient's intracardiac electrogram 714 is a reference for the acquired impedance signals. Ideally, the peak of the resulting impedance curves should occur at a synchronous point in time for symmetrically stimulated myocardial segments.

As shown in FIG. 7, Z1(t) 710 starts with ventricular depolarization, and has a peak value Z1p disposed between the AoVo, the time that the aortic valve opens, and AoVc, the time that the aortic valve closes, referenced to the EGM signal 714. Some electrodes will generate impedance signals, such as Z1(t) 710, where timing of aortic valvular events by notches on the upslope, NU, and downslope, ND, of the impedance signal can be identified.

Impedance curve Z2(t) 712 has a peak value Z2p at a time after the peak value Z1p of impedance curve Z1(t) 710. Ideally, the peak of the resulting impedance curves Z1(t) 710, Z2(t) 712 should occur at a synchronous point in time between aortic valve opening and aortic valve closure. However, in a heart with dysynchrony, the two peaks can be shifted significantly.

Impedance parameters that can be analyzed to determine whether the patient has the possibility of dysynchrony include, but are not limited to the time of onset of Z(t), the time of peak dZ/dt, the time of Z(peak), Z(peak), dZ′/dt (the first derivative of the impedance waveform), dZ″/dt (the second derivative of the impedance waveform), systolic integrals of Z(t)/dt, diastolic integrals of Z(t)/dt, identification of valvular events from the impedance waveform, changes in impedance waveform morphology, comparison of the impedance waveform to a normal waveform template, and the like.

In block 414, the microcontroller 60 determines whether the parameter is indicative of dysynchrony. When the parameter does not indicate dysynchrony, the process 400 moves to block 410 to receive additional signals from the sensors 22, 26, 27, 28, 32, 34, 36, 38.

When the parameter indicates dysynchrony, the process 400 moves to block 416, where an indication of dysynchrony is stored in the device 10. In an embodiment, the indication of dysynchrony or an alert is stored in the memory 94. In another embodiment, the device 10 enunciates the indication of dysynchrony. Examples of enunciation comprise audible alarms, vibration alarms, and the like.

In an embodiment, the indication can be uploaded from the device 10 to the external device 102 by the attending medical personnel. The alert notifies the following physician to consider upgrading the device 10 to one that has resynchronization capabilities.

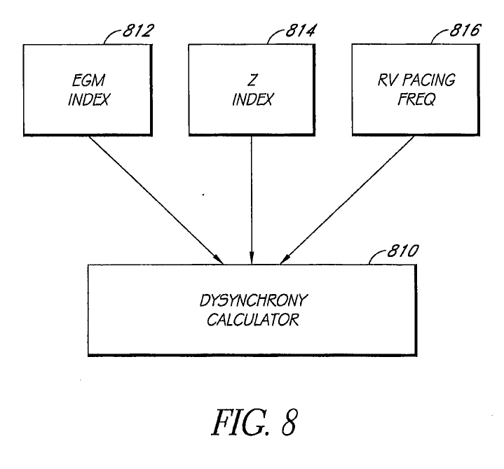

FIG. 8 is a block diagram illustrating the dysynchrony calculator 810, according to an embodiment of the invention. The dysynchrony calculator 810 receives EGM data 812, impedance data 814, and pacing data from the device 10, analyzes the data for the possibility of dysynchrony, and determines whether the patient has the possibility of dysynchrony. In an embodiment, the dysynchrony calculator 810 is incorporated into the device 10, as illustrated in FIG. 2, and can be programmed by the external device 102. In another embodiment, the dysynchrony calculator 810 is incorporated into the external device 102.

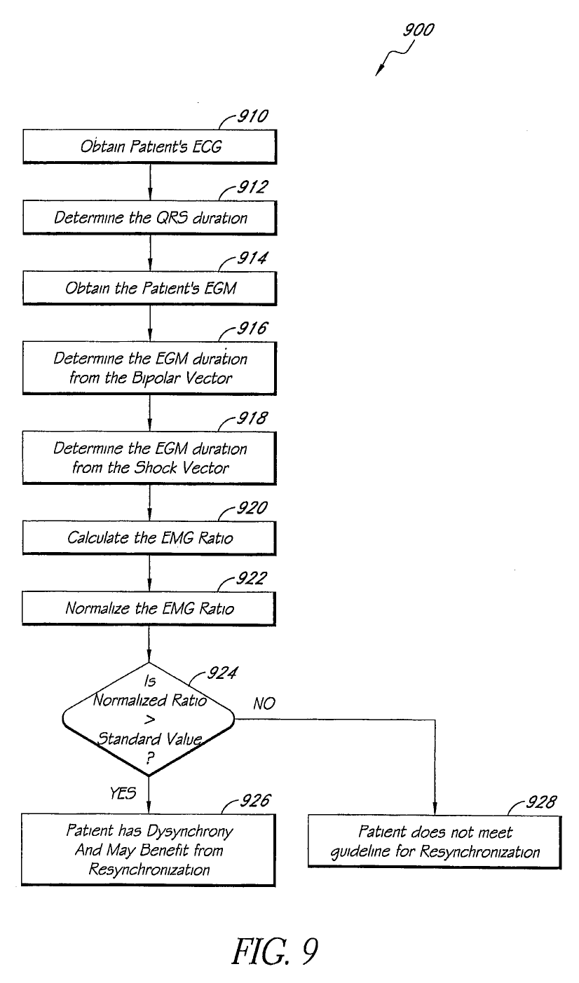

Assessing the patients' intrinsic conductivity patterns using intracardiac electrograms obtained with intracardiac stimulation devices 10 along with surface ECGs can identify patients with dysynchrony. FIG. 9 illustrates an embodiment of a process 900 to identify patients with dysynchrony using EGM and ECG data.

In block 910, the patient's ECG is obtained and in block 912, the duration of the patient's QRS complex from the ECG data is determined.

In block 914, the device 10 receives at least one signal indicative of the patient's intracardiac electrogram using at least one of the electrodes 22, 26, 27, 28, 32, 34, 36, 38 coupled to at least one of the sensing circuits 82, 84. The patient's EGM can be obtained from multiple electrode pairs. In an embodiment, device 10 senses and records the patient's EGM measured from the bipolar vector 510.

In another embodiment, the device 10 senses and records patient's EGM measured from the shock vector 512. The microprocessor 60 stores the EGM signal data in the memory 94.

In block 916, the dysynchrony calculator 810 determines a duration of the EGM signal from the bipolar vector 510, and in block 918, determines a duration of the EGM signal from the shock vector 512.

The ratio of the duration of the EGM signal from the bipolar vector 10 to the duration of the EGM signal from the shock vector 512 is calculated in block 920 to produce the patient's EGM ratio.

In block 922, the dysynchrony calculator 810 normalizes the patient's EGM ratio by dividing the EGM ratio by the duration of the patient's QRS complex to produce a normalized ratio.

In block 924, the dysynchrony calculator 810 compares the normalized ratio to a threshold. In an embodiment, the threshold is between approximately 0.004 and 0.010, and preferably between approximately 0.005 and 0.008.

In an embodiment, when the normalized ratio is less than the threshold, the process 900 moves to block 928, where the dysynchrony calculator 810 determines that the patient may not benefit from resynchronization therapy.

In another embodiment, when the normalized ratio is greater than the threshold, the process 900 moves to block 926, where the dysynchrony calculator 810 determines that the patient may benefit from cardiac resynchronization therapy. In an embodiment, the dysynchrony calculator 810 indicates an alert, which can be a flag stored in memory that is retrieved by the attending medical personnel. In another embodiment, the alert can be an audible indication, which alerts the patient and the attending physician that the patient is potentially subject to heart dysynchrony. In a further embodiment, the implanted device may have the functionality to determine and implement an appropriate resynchronization therapy for the patient. Upon retrieval of the alert, the attending physician can evaluate the patient's candidacy for cardiac resynchronization therapy (CRT).

In an embodiment where the microprocessor 60 comprises the dysynchrony calculator 810, the patients EGM signal data can be stored in the memory 94 and the patient's ECG data can be downloaded from the external device 102 to the device 10. In this embodiment, the microprocessor 60 performs the calculation to determine the possibility of dysynchrony and the microprocessor 60 indicates the possibility of dysynchrony in the memory 60. The microprocessor 60 can indicate the possibility of dysynchrony by setting a flag in the memory 94 to be retrieved by the attending physician, initiating an audible alert, or the like. Upon retrieval of the alert, the attending physician can evaluate the patient's candidacy for cardiac resynchronization therapy (CRT).

In another embodiment, the patient's EGM signal data can be uploaded from the device 10 through the telemetry circuit 100 and the link 104 to the external device 102. In this embodiment, the external device 102 comprises the dysynchrony calculator 810, performs the calculations to determine the possibility of dysynchrony, and indicates the possibility of dysynchrony to the attending medical personnel. The external device 102 can indicate the possibility of dysynchrony by setting a flag in the memory 130 to be retrieved by the attending physician, initiating an audible alert, or the like. Upon retrieval of the alert, the attending physician can evaluate the patient's candidacy for cardiac resynchronization therapy (CRT).

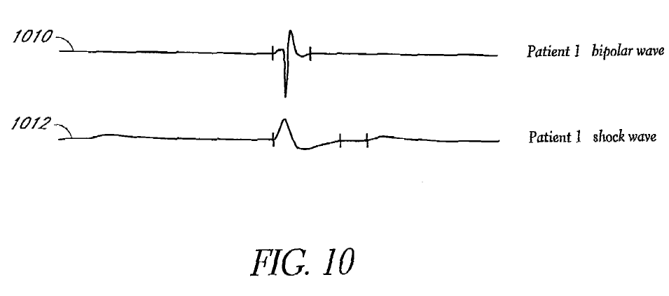

The following example further illustrates the process 900. FIG. 10 illustrates graphical representations of Patient 1's EGM data. EGM 1010 represents Patient 1's EGM signal recorded from the bipolar vector 510 and EGM 1012 represents Patient 1's EGM signal recorded from the shock vector 12. The x-axis indicates time in seconds and is measured at approximately 100 mm/second.

As illustrated in FIG. 10, the duration of the EGM signal 1010 is approximately 120 msec and the duration of the EGM signal 1012 approximately 218 msec. The duration of the QRS complex for Patient 1's measured from Patient 1's ECG, is approximately 166 msec. Patient 1's EGM ratio, the ratio of the duration of the EGM signal from the bipolar vector 510 to the duration of the EGM signal from the shock vector, is 120 msec/218 msec, or approximately 0.55.

Patient 1's normalized ratio, the EGM ratio divided by the duration of the surface QRS complex width is 0.55/166 or approximately 0.00331.

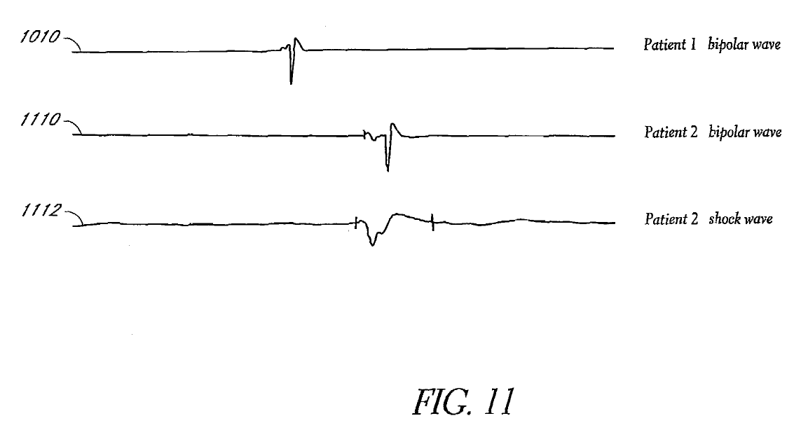

Likewise, FIG. 11 illustrates graphical representations of Patient 2's EGM data. EGM 1110 represents Patient 2's EGM signal recorded from the bipolar vector 510 and EGM 1112 represents Patient 2's EGM signal recorded from the shock vector 512. The x-axis indicates time in seconds and is measured at approximately 100 mm/second.

As illustrated in FIG. 11, the duration of the EGM signal 1110 is approximately 114 msec and the duration of the EGM signal 1112 approximately 170 msec. The duration of the QRS complex for Patient 2 is approximately 104 msec, as measured from Patient 2's ECG. Patient 2's EGM ratio, the ratio of the duration of the EGM signal from the bipolar vector 510 to the duration of the EGM signal from the shock vector, is 114 msec/170 msec or approximately 0.67.

Patient 2's normalized ratio, the EGM ratio divided by the duration of the surface QRS complex width is 0.67/104 or approximately 0.00644.

The data is summarized in the following table.

| Patient | CHF Class | QRS | EGM Ratio | Normalized Ratio |
|---|---|---|---|---|
| 1 | Class I/II | 166 msec | 0.55 | 0.00331 |
| 2 | Class III | 104 msec | 0.67 | 0.00644 |

In this example, Patient 1 who has no CHF symptoms and a wide QRS complex has less of a differential in intracardiac EGM width than Patient 2 who has CHF symptoms and a narrow surface QRS signal. In an embodiment, guidelines based on the surface ECG data indicate that patients having a QRS complex of greater than 120 msec would benefit from cardiac resynchronization therapy. Thus, Patient 1 would be a candidate for cardiac resynchronization therapy and Patient 2 would not, using the surface ECG data. However, the normalized EGM ratio shows that Patient 2 has a greater differential in intracardiac width than Patient 1 and thus, Patient 2 has a greater dysynchrony than Patient 1. Even though Patient 2 does not have a wide QRS complex, Patient 2 is a candidate for cardiac resynchronization therapy.

FIG. 11 further comprises the EGM signal 1010 from the bipolar vector 510 of Patient 1 shown relative to the EGM signal 1110 from the bipolar vector 510 of Patient 2. As illustrated in FIG. 11, there is a greater difference in EGM onset in Patient 2 than Patient 1 for these vectors, which further indicates dysynchronous conduction properties in Patient 2.

Morphology, template or other analyses of intracardiac EGM data can identify the large number of patients with conventional defibrillators, pacemakers, implanted cardiac devices, and the like that would benefit from a device upgrade. This monitoring can be evaluated by the physician in follow-up visits thus identifying patient candidacy for a device upgrade to a unit with resynchronization capabilities. A software package which analyzes intracardiac EGM data and impedance measurements can be downloaded into currently implanted cardiac device systems.

Further, once the resynchronization process is implemented, further analyses of the intracardiac electrogram data and impedance measurements can improve the resynchronization.

While certain embodiments of the inventions have been described, these embodiments have been presented by way of example only, and are not intended to limit the scope of the inventions. Indeed, the novel methods and systems described herein may be embodied in a variety of other forms; furthermore, various omissions, substitutions, and changes in the form of the methods and systems described herein may be made without departing from the spirit of the inventions. The accompanying claims and their equivalents are intended to cover such forms or modifications as would fall within the scope and spirit of the inventions.

What is claimed is:

1. An implantable cardiac stimulation device comprising:
at least one lead for providing electrical stimulation to the heart of a patient;
at least one sensor that detects electrical signals indicative of the depolarization of the heart of the patient; and
a controller that is adapted to be implanted within the patient;
wherein the controller receives signals from the at least one sensor and further induces the lead to provide therapeutic electrical stimulation to the heart of the patient;
wherein the controller periodically evaluates the signals from the sensor and determines if at least one parameter of the signal is indicative of the patient being potentially subject to heart dysynchrony, wherein such determination is made by comparing the received signals to stored signals developed from other patients having dysynchrony; and
wherein the controller, upon determining that the parameter of the signal indicates that the patient is potentially subject to heart dysynchrony, records an indication thereof for subsequent communication to treating medical personnel.

2. The device of claim 1, wherein the at least one lead comprises a plurality of leads that are positioned in proximity to the walls of the heart of the patient.

3. The device of claim 2, wherein the plurality of leads are positioned within at least two chambers of the heart of the patient.

4. The device of claim 1, wherein the at least one sensor detects electrical signals comprising an intra-cardiac electrogram.

5. The device of claim 4, wherein the controller assess a duration parameter of the intra-cardiac electrogram and compares the duration parameter to a criterion to determine whether the sensed signal is indicative of potential dysynchrony of the patient.

6. The device of claim 5, wherein the at least one sensor comprises sensors attached to the at least one leads and wherein the at least one lead comprises an atrial lead and at least one ventricle lead and wherein the duration parameter comprises a parameter associated with the time between atrial depolarization and the QRS complex of the latest depolarization of all detected in an intra-cardiac electrogram.

7. The device of claim 6, wherein the duration parameter comprises the time between the atrial depolarization and the terminal portion of the latest occurring EGM depolarization.

8. The device of claim 7, wherein the terminal portion is defined as the time where the change in voltage over the change in time is at a minima.

9. The device of claim 5, wherein the duration parameter comprises a parameter associated with the duration of the QRS complex of the intra-cardiac electrogram.

10. The device of claim 1, wherein the at least one sensor detects impedance signals within a chamber of the heart during at least one cycle of the heart and wherein the controller evaluates the impedance signals to determine whether the patient is potentially subject to heart dysynchrony.

11. The device of claim 1 further comprising memory in communication with the controller, wherein the controller records the indication in the memory.

12. The device of claim 11 further comprising telemetry circuitry configured to communicate with an external device the indication, wherein the treating medical personnel receive the indication from the external device.

13. A method for using an implantable cardiac stimulation device comprising:
providing electrical stimulation with at least one lead to the heart of a patient;
detecting electrical signals with at least one sensor indicative of the depolarization of the heart of the patient;
receiving the signals with a controller adapted to be implanted within the patient and providing therapeutic electrical stimulation to the heart of the patient based at least in part on the received electrical signals;
periodically evaluating the signals with the controller and determining with the controller whether at least one parameter of the signal is indicative of the patient being potentially subject to heart dysynchrony by comparing the received signals to stored signals developed from other patients having dysynchrony; and
upon determining that the parameter of the signal indicates that the patient is potentially subject to heart dysynchrony, recording with the controller an indication thereof for subsequent communication to treating medical personnel.

14. The method of claim 13, wherein providing electrical stimulation with the at least one lead comprises providing electrical stimulation with a plurality of leads that are positioned in proximity to the walls of the heart of the patient.

15. The method of claim 13, wherein providing electrical stimulation comprises providing pacing and delivering high voltage signals to the heart.

16. The method of claim 13, wherein the controller is positioned within a casing that can also form an electrode for the sensor that detects electrical signals indicative of the depolarization of the heart of the patient.

17. The method of claim 16 further comprising selecting at least one vector indicative of the depolarization of the heart of the patient to be sensed by the sensor, wherein each of the plurality of leads and the casing define a plurality of the vectors.

18. The method of claim 13, wherein detecting electrical signals comprises detecting an intra-cardiac electrogram.

19. The method of claim 13, wherein detecting electrical signals comprises sensing impedance signals of the heart during at least one cycle of the heart and periodically evaluating the signals comprises evaluating the impedance signals to determine whether the patient is potentially subject to heart dysynchrony.

20. The method of claim 13 further comprising communicating the indication to an external device, wherein the treating medical personnel receive the indication from the external device.
